(19) United States
(12) Patent Application Publication
(10) Pub. No.: US 2003/0134816 A1
(43) Pub. Date: Jul. 17, 2003

(54) Therapeutic compounds

(76) Inventors: Gabriele Braun, Bristol (GB); Nicol McKechnie, Bristol (GB)

Correspondence Address: SCHWEGMAN, LUNDBERG, WOESSNER & KLUTH, P.A., P.O. BOX 2938, MINNEAPOLIS, MN, 55402, US

(21) Appl. No.: 10/300,575

(22) Filed: Nov. 20, 2002

Related U.S. Application Data
(63) Continuation of application No. PCT/GB01/02397, filed on May 30, 2001.

(30) Foreign Application Priority Data
May 30, 2000 (GB) 0013105.2

(51) Int. Cl. A61K 48/00; C07H 21/04; A61K 39/395; C07K 16/40

(52) U.S. Cl. 514/44; 530/388.26; 536/23.2; 424/146.1

(57) ABSTRACT

The present invention relates to a compound capable of modulating the activity and/or expression of hr44, for use in therapy.

### RELATED APPLICATIONS

This application is a continuation under 35 U.S.C. 111(a) of PCT/GB01/02397 filed May 30, 2001 and published in English as WO 01/92521 A1 on Dec. 6, 2001, which claims priority from Great Britain application 0013105.2 filed May 30, 2000, which applications and publication are incorporated herein by reference.

### BACKGROUND

Hr44 has been cloned from human retinal tissue (Braun G. et al (1995) J. Exp. Med. 182:1121-1132—Accession number X91103 or Q15333). The bovine homologue of hr44 has been identified by Frank et al., and deposited in the EMBL/GenBank/DDBJ databases on Dec. 7, 2000 (Prim. Accession CAC16354). Analysis of the primary structure of the protein indicates that hr44 is a type I membrane protein with an amino terminal extracytosolic domain, a transmembrane domain, and a very short carboxy-terminal cytosolic fragment consisting of basic residues. Hr44 is also soluble in aqueous solution and has been localised in the cell cytoplasm and in the nucleus. Hr44 has calcium binding sites, sites for phosphorylation by tyrosine kinase, by protein kinase C and casein kinase II.

Hr44 is expressed in metabolically active cells and tissues, e.g. heart, skeletal muscle; some neurons and many epithelia, including skin, ciliary epithelium, renal proximal tubule, and ependyma; in fat storage tissues, e.g. liver and adipose tissue. It is highly expressed in steroidogenic tissue, e.g., adrenals and testis and other glandular tissue like the haderian gland and pancreas. It occurs in bile, plasma and pancreatic juice. Hr44 appears to be expressed as tissue specific versions with tissue specific levels of expression.

Hr44 has been investigated as an autoantigen in the development of potential ocular autoimmune pathology caused by the tropical filarial infection onchocerciasis (McKechnie et al (1997) J. Infect. Dis. 176:1334-43). However, no biological function of hr44 has been determined previously.

### SUMMARY OF THE INVENTION

The present invention relates to compounds for use in therapy. In particular, the present invention relates to therapeutic compounds capable of modulating the activity and/or expression of hr44.

The present invention also relates to a method for screening for such therapeutic compounds and to various therapeutic applications of the compounds.

The present invention also relates to methods for monitoring disease progression or for diagnosing a disease based on the detection or measurement of hr44 expression.

The present inventors have shown that hr44 interacts with a number of cytoplasmic and nuclear proteins. In particular, hr44 has been shown to interact with proteins which are involved in the following metabolic activities:

i) the regulation of enzymes of fatty acid metabolism;

ii) the transport of prostaglandins and/or related substances;

iii) the altered glycolytic profile characteristic of tumours; and

iv) transcriptional regulation, including the regulation of genes involved in i, ii and iii.

Hence, compounds capable of modulating the activity and/or expression of hr44 have applications for therapy of diseases, in particular for diseases associated with alterations in glycolysis, fatty acid formation/transport and metabolism, and transport of prostaglandin/prostaglandin-like molecules. All of the proteins which have been shown to interact with hr44 have been implicated in oncogenic alterations in metabolism. Therefore, the compounds are particularly suited for use in the prevention and/or treatment of cancer.

Moreover, it has been determined that hr44 exists in a plurality of forms, which fall into two basic categories: hr44 molecules associated with the cytoplasmic membrane, the endoplasmic reticulum or endosomal compartments, and hr44 associated with the nucleus. These various forms of hr44 are generated by alternative splicing of the hr44 transcript. A specific sequence that targets hr44 to endosomal compartments has been identified.

Furthermore, hr44 is differentially glycosylated according to its source. Differences in glycosylation patterns are particularly associated with cancer, especially colonic adenomas and carcinomas.

In a first aspect, therefore, the present invention relates to novel therapeutic compounds. In particular, the present invention relates to compounds capable of modulating the activity, expression and/or subcellular localisation of hr44.

The present inventors have shown that hr44 interacts with the following proteins: M2-type and M1-type pyruvate kinase; the heterogeneous nuclear ribonucleoprotein hnRNPE1; protein YP4 (a novel protein identified by the inventors, which shares some identity with prostaglandin transporters and organic anion transporters); and fibrillarin. In a preferred embodiment, therefore, compounds according to the first aspect of the invention are capable of modulating the interaction of hr44 with M1-type/M2-type pyruvate kinase and/or hnRNPE1 and/or YP4 and/or fibrillarin.

The present inventors have also shown that hr44 has the capacity to homodimerise.

The compounds of the first aspect of the invention have a number of therapeutic and prophylactic applications. Hence, in a second aspect, the present invention provides a pharmaceutical composition comprising a compound of the first aspect of the invention. The third aspect of the invention relates to methods of treating and/or preventing a disease using a compound of the first aspect of the invention or a pharmaceutical composition according to the second aspect of the invention.

In a fourth aspect, the present invention relates to a method for identifying a compound useful in therapy. In particular such a compound may be identified by screening a plurality of compounds and selecting a compound which binds to hr44, or modulates the activity or expression of hr44.

The present inventors have also shown that the presence of hr44 is characteristic of a number of diseases, and that the level of hr44 is indicative of the stage of the disease. Hence the present invention also relates to diagnostic methods, and methods of monitoring the progression of a disease, based on the detection or measurement of hr44 expression.

In particular, the present invention provides antibodies capable of specific detection of differentially glycosylated forms of hr44 in tumours such as colonic adenomas and carcinomas.

The present inventors have identified a novel protein (termed “YP4”) which binds hr44, and obtained its partial cDNA and amino acid sequences. The present invention also relates to novel polynucleotides and polypeptides. In particular, the present invention relates to a polynucleotide comprising the partial YP4 cDNA sequence (SEQ ID NO: 1), and a polypeptide comprising the partial YP4 amino acid sequence (SEQ ID NO:2).

### DETAILED DESCRIPTION OF THE INVENTION

In a first aspect, the present invention relates to a compound capable of modulating the activity and/or expression of hr44.

Compounds

The term “compound” refers to a chemical compound (naturally occurring or synthesised), such as a biological macromolecule (e.g., nucleic acid, protein, peptide, carbohydrate or organic molecule), or an extract made from biological materials such as bacteria, plants, fungi, or animal (particularly mammalian) cells or tissues, or even an inorganic element or molecule.

Preferably a compound according to the first aspect of the invention is identifiable by screening a library of candidate compounds. The screening method of the fourth aspect of the invention is particularly suited to screening libraries of compounds in multi-well plates (e.g., 96-well plates), with a different test compound in each well. In particular, the library of candidate compounds may be a combinatorial library. A variety of combinatorial libraries of random-sequence oligonucleotides, polypeptides, or synthetic oligomers have been proposed and number of small-molecule libraries have also been developed. Combinatorial libraries of oligomers may be formed by a variety of solution-phase or solid-phase methods in which mixtures of different subunits are added stepwise to growing oligomers or parent compound, until a desired oligomer size is reached (typically hexapeptide or heptapeptide). A library of increasing complexity can be formed in this manner, for example, by pooling multiple choices of reagents with each additional subunit step. Alternatively, the library may be formed by solid-phase synthetic methods in which beads containing different-sequence oligomers that form the library are alternately mixed and separated, with one of a selected number of subunits being added to each group of separated beads at each step. Libraries, including combinatorial libraries are commercially available form pharmaceutical companies and speciality library suppliers.

The identity of library compounds with desired effects on hr44 activity and/or expression can be determined by conventional means, such as iterative synthesis methods in which sublibraries containing known residues in one subunit position only are identified as containing active compounds.

Modulation

“Modulation” refers to the capacity to either increase or decease the activity and/or expression of hr44 by at least 10%, 15%, 20%, 25%, 50%, 100% or more; such increase or decrease may be contingent on the occurrence of a specific event, such as activation/suppression of a signal transduction pathway, and/or activation or inactivation of cellular proteins and/or translocation of cellular proteins. Such events may be selectively manifest in particular cell types.

Modulation of Hr44 Activity

The present inventors have shown that hr44 interacts with YP4, a novel protein identified and named by the inventors. YP4 has 54% homology and 36% identity with known prostaglandin transporters (PGTs). The known PGTs have approximately the same percentage homologies and identities when compared with each other. These molecules are integral membrane proteins, presumed to mediate release, uptake, epithelial transport or metabolic clearance of organic anions (such as prostaglandins and derivatives, conjugated steroids and thyroid hormones—Schuster V L, 1998, Annu. Rev. Physiol. 60:221-242). Hr44 is therefore thought to be involved in the regulation and/or transport of prostaglandins and/or related substances.

The present inventors have also shown that hr44 interacts with hnRNPE1 (heterogeneous nuclear ribonucleoprotein). HnRNPE1 represses the formation of lipoxygenase 15-LOX-1, by acting as a translational silencer (Ostareck D H et al, 1997, Cell 89:597-606). 15-LOX-1, like 5-LOX and 12-LOX, belongs to a family of lipoxygenases that metabolise fatty acids like linoleic acid and arachidonic acid. One of the immediate metabolites of 15-LOX-1 is 13-HODE (hydroxyoctadecadienoic acid).

Interaction of hr44 with YP4 and hnRNPE1 may constitute a regulatory mechanism of fatty acid metabolism and transport. Without wishing to be bound by theory, the present inventors propose that hr44 inhibits fatty acid metabolism by interacting and stabilising the hnRNPE1 suppressor effect on lipoxygenase formation. This reduces the intracellular pool of 13-S-HODE and possibly increases the pool of arachidonic acid, which is the precursor of prostaglandins. By interacting with YP4, hr44 affects the transport of those molecules across membranes (uptake as well as release from cells).

The present inventors have also shown that hr44 interacts with M1-type and M2-type pyruvate kinase. Pyruvate kinases are key enzymes in glycolysis and the M2-type is predominantly expressed in tumerogenic tissue (Yamada K and Noguchi T, 1999, Biochemical Journal 337:1-11). M2-type pyruvate kinase mediates degradation of pyruvate to lactate. It exists in two forms, as a low-affinity dimeric form and a tetrameric form with higher substrate specificity (Eigenbrodt E, et al, 1997, Anticancer Res. 17:3153-3156).

The inventors have shown that hr44 is highly expressed in colon cancer tissue. Interaction of hr44 with M2-type pyruvate kinase may stabilise the dimer form of the enzyme leading to an elevated phosphometabolite pool. Pyruvate can be incorporated into fatty acids via acetyl CoA pathway (2) and hr44 may thus contribute to enhanced lipogenesis.

It is therefore proposed that hr44 contributes to an increase of fatty acid concentrations in two ways:

i) by regulation of metabolic pathways central to the switch points between glycolysis and fatty acid formation; and

ii) by inhibiting pathways of fatty acid metabolism.

The present inventors have also shown that hr44 interacts with fibrillarin. Fibrillarin may also be described as a heterogeneous nuclear ribonucleoprotein as it is associated with RNA complexes. Like other ribonucleoproteins (e.g. hnRNPE1), it is implicated in gene expression at the translational level. Fibrillarin can relocate from the nucleus to the cytoplasm in response to heat shock (shown with the fibrillarin homologue NOP1p of Drosophila melanogaster, (Liu Y, et al, 1996, EMBO JOURNAL 15:6750-6757)) and potentially other signals. Hr44 is thought to take part in regulation of gene expression via its interaction with fibrillarin. Thus hr44 is thought to be involved in the expression of genes other than 15-LOX1 which are associated with fatty acid metabolism.

Hence, the compound may affect the activity of hr44 by modulating its capacity to interact with YP4, and/or hnRNPE1 and/or pyruvate kinase and/or fibrillarin. The compound may thus affect lipogenesis, fatty acid metabolism and signalling events.

A compound capable of modulating the activity of hr44 may act as an agonist or antagonist of the activity. An antagonistic compound should bind hr44 with a higher binding affinity in order to successfully compete for hr44 interaction with its physiological or “in vivo” binding partners. As hr44 has been demonstrated to interact with itself, interruption or stabilisation of the homodimer formation could inhibit or abrogate hr44 function.

Hr44 is a type I protein associated with the cytoplasmic membrane. A green fluorescent protein (gfp) tagged version of hr44 accumulates in endosomes as it can be co-localised with the transferrin receptor, a marker of the endosomal compartment. A version of hr44, generated by alternative splicing, can be localised in the nucleus of some tissue (particularly ducted glands) and a gep-tagged alternatively spliced version of hr44 localises to the nucleus of transfected cells.

The membrane associated version of hr44 is highly glycosylated and is in many ways similar to some adhesion molecules and glue proteins and also to a family of type I membrane proteins collectively named LAMPS (lysosomal-associated membrane proteins) which have been implicated in tumour development. Tumour differentiation and metastasis are associated with drastic alterations of the ratio of splice variants and glycosylation of such tumour associated molecules.

Most of the available monoclonal antibodies to hr44 were raised against hr44 expressed in a bacterial system and therefore lacking any eukaryotic post-translational modifications. One antibody according to the invention fails to recognise normal colonic epithelium but identifies hr44 in colonic adenomas. This implies differential glycosylation of hr44 in tumours. A tumour specific glycosylation variant of hr44 therefore exists, which is indicated by the finding of a restricted recognition pattern by a monoclonal antibody in accordance with the invention.

Hr44 is also markedly upregulated in colonic adenomas and increased expression correlates well with increasing size of the tumour. This has been demonstrated with a monoclonal antibody which recognises hr44 in a wider variety of tissues. Hr44 is also present in adenocarcinomas.

Expression of hr44 is hypoxically regulated. This explains the positive association of level of expression with solid tumour size. It also explains the distribution of hr44 in other tissues. Müiller cells, fast (large) muscle fibres, and solid tumours all function under hypoxic conditions (with incomplete glycolysis). The upregulation of hr44 in solid tumours contributes to the survival of cells in hypoxic environments with regard to adaptation to altered metabolic demands and also with regard to conferring radiation resistance.

Radiation sensitive tumours do not display any hr44 expression; for example, retinoblastoma is hr44-negative. Accordingly, combined therapies including hr44 downregulation and radiation treatment are effective in the therapy of normally radiation-resistant tumours.

In addition, hr44 plays an important role in the extrusion of chemotherapeutic agents, an undesirable property of tumour cells. Hr44 is a component of the exocytotic pathways (endoplasmic reticulum and Golgi), on the cell surface, and in endocytotic vesicles targeted to endosomes. Endosomes and recycling vesicles are important in the regulation of intracellular pH. Integral membrane pumps normally acidify endosomes and recycling vesicles, forming a sink for H+ions. An important consequence of the acidification of these vesicles is their uptake of weakly basic chemotherapeutic drugs (e.g. Anthracyclines and vinca alkaloids). These concentrate in acidic organelles and their subsequent extrusion from the cell through the secretory pathway may contribute to chemotherapeutic resistance.

Modulation of Hr44 Expression

There are numerous mechanisms known in the art by which the expression of a protein may be modulated.

The expression of a protein may be increased in a particular cell by expression of the protein itself from a heterologous promoter. For example, the cell can be transfected with a vector comprising the gene, which expresses the protein independently from expression of the endogenous gene. Alternatively, the activity or expression of one or more of the cellular components involved in controlling transcription of the gene can be modulated.

The expression of a protein can be reduced by directly interfering with transcription and/or translation of the gene, for example, by the use of antisense technology. In this respect, the compound may be a nucleic acid sequence capable of hybridising with the hr44 mRNA sequence. Candidate compounds useful in the inhibition of hr44 expression can thus be designed based on the nucleic acid sequence of hr44.

Northern Blot analysis indicates that there are tissue specific variants of hr44, appearing in normal tissues, and one variant common to a variety of carcinomas. In a preferred embodiment, the compound is capable of interacting specifically with a particular tissue-specific variant of hr44. Preferably, this tissue specific variant is specific for one or more tumours.

In one embodiment of the first aspect of the invention, the compound is an antibody.

Antibodies

Antibodies may be produced by standard techniques, such as by immunisation with hr44 or a fragment thereof, or by using a phage display library.

For the purposes of this invention, the term “antibody”, unless specified to the contrary, includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies, for example as described in EP-A-239400. Neutralising antibodies, i.e., those which inhibit biological activity of the substance amino acid sequences, are especially preferred for diagnostics and therapeutics.

Therapy

The compound of the present invention may be suitable for use in therapy.

The term “therapy” means the treatment or prevention of a medical condition. In particular, therapy relates to treatment or prevention of a condition by the administration of a therapeutic or prophylactic composition.

Pharmaceutical Compositions

In a second aspect, the present invention provides a pharmaceutical composition comprising a compound of the first aspect of the invention. The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s), and other carrier agents that may aid or increase the viral entry into the target site (such as for example a lipid delivery system).

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

In a third aspect, the present invention relates to a method for treating and/or preventing a disease, which comprises the step of administering a compound according to the first aspect of the invention to a subject.

Diseases

The compounds of the present invention are particularly suited for use in therapeutic applications for diseases associated with an irregularity in one or more of the following: glycolysis, lipogenesis, fatty acid metabolism/transport and prostaglandin formation/transport.

Dietary fatty acids (predominately linoleic acid and arachidonic acid) are essential in the provision of metabolic energy and contractile energy of the heart and skeletal muscle and for the synthesis of cellular triglycerides stored in adipocytes of fatty tissue and in hepatocytes.

Most cells are dependent on blood derived fatty acids for their oxidative energy needs. Fatty acids are essential for synthesis of phospholipids, prostaglandins, cholesterol esters and steroid hormones. Fatty acids also function as signal transducers by modulating activity of ionic channels, synaptic transmission and expression of genes involved in lipid metabolism and cell differentiation. Fatty acids are metabolised through pathways involving lipoxygenases (LOX, including 5-LOX, 12-LOX and 15-LOX) and cyclooxygenases.

There is strong experimental evidence that enzyme activity of 15-LOX1 is involved in vascular disease (Cornicelli J A and Trivedi B K, 1999, Current Pharmaceutical Design 5:11-20; Bailey J M et al, 1995, Atherosclerosis 113:247-258). Chemical inhibitors of 15-LOX1 have been shown to prevent atherosclerotic lesions in animal models. Atherosclerosis is the cause of major diseases like ischemic heart disease, stroke and myocardial infarction.

Hr44 interacts with hnRNPE1, which represses the formation of 15-LOX. Hence compounds capable of modulating the activity and/or expression of hr44 are useful for the treatment of vascular diseases, in particular those associated with atherosclerosis. The compounds of the present invention may be used to treat or prevent vascular diseases such as ischemic heart disease, stroke and myocardial infarction.

The compounds of the present invention may also be used to treat certain type of cancers as fatty acid metabolites play also a crucial role in the promotion of tumours. This is demonstrated by the fact that chemical inhibitors (non-steroidal anti-inflammatory drugs) of prostaglandin formation (derivative of the fatty acid arachidonic acid) are the most powerful preventive agents in animal models and can reduce the incidence of colorectal cancer in man by half. The lipoxygenase and cyclooxygenase pathways of arachidonic acid metabolism have been implicated in tumour promotion and malignant progression.

Human colon cancers appear to be associated with up-regulation of cyclooxygenase 2. Pancreatic cancers are associated with up-regulation of 5-LOX and 12-LOX. 15-LOX1 has been shown to be down-regulated in colon cancer (Fosslien E, 2000, Annals of Clinical & Laboratory Science, 30: 3-21; Xian-Zhong Ding et al, 1999, Biochem. Biophys. Res. Communications 266:392-399; Shureiqi I et al, 1999, Carcinogenesis, 20:1985-1995). A metabolic product of 15-LOX1 is 13-S-HODE. 13-S-HODE suppresses cell proliferation and induces apoptosis in transformed colonic epithelial cells (Shureiqi I et al, 1999, Carcinogenesis 20:1985-1995). Lack of this product may thus promote development of certain cancers.

In addition, neoplastic cells have special requirements and their development is associated with high rates of glucose uptake and glycolysis (Chi V Dang and Lemenza G L, 1999, TIBS 24:68-72). Glycolysis is required to cover the high energy demand of increased cell proliferation and the glycolytic phosphometabolites are needed for the biosynthesis of nucleic acids, phospholipids etc. One of the key enzymes regulating the phosphometabolite pool is pyruvate kinase. There are four types of pyruvate kinase, M1-, M2-, L- and R-type. M1 appears in differentiated tissues like skeletal muscle, heart and brain, L in gluconeogenic tissues like liver, R in erythrocytes and haematopoietic tissues. M2-type is predominant in neoplastic tissue (Yamada K and Noguchi T, 1999, Biochem J 337:1-11). Oncogenic alteration of metabolism in response to hypoxia is associated with progressive replacement of M1-type pyruvate kinase by the M2-type and the M2 isoenzyme is regarded a marker for tumerogenic transformation (Guminska M et al, 1997, Acta Biochimica Polonica 44:711-724). In tumours the low-affinity dimeric form of M2-type pyruvate kinase is accumulated (Eigenbrodt E, et al, 1997, Anticancer Res. 17:3153-3156). The dimer form has recently been reported to be stabilised by E7 oncoprotein of papillomavirus (HPV) and is thought to contribute to cell transformation (Zwerschke W, et al, 1999, Proc. Natl. Acad. Sci, USA 96:1291-1296).

Hr44 is overexpressed in tumours and the present inventors propose that complex formation with the dimer form of M2 pyruvate kinase allows biosynthetic activity, by which pyruvate can be incorporated into fatty acids via the acetyl CoA pathway, increasing the pool of prostaglandin precursors. Lipogenesis is much enhanced in tumours of the breast (Kuhajda F, et al, 1994, Proc. Natl. Acad. Sci USA 91:6279-6383), prostate (Shurbji M, et al, 1996, Hum, Pathol. 27:917-921), colorectal cancers (Reston M et al, 1992, Lab. Invest. 66:47) and cancers of the ovaries (Gansler T, et al, 1997, Hum. Pathol. 28:686-692.) and endometrium (Pizer E, et al, 1996, Cancer Res. 56:2745-2747). Lipogenesis is an established marker of poor prognosis. Hr44 is also thought to contribute to negative translational regulation of 15-LOX1 and to depletion of 13-HODE with the effect of reduced apoptosis and promotion of tumour growth.

In the method of the third aspect of the invention, the subject may be a human or animal subject. Preferably the subject is a mammalian subject, more preferably a human subject. Typically, a physician will determine the actual dosage which will be most suitable for an individual subject and it will vary with the age, weight and response of the particular patient.

Screening Methods

In a fourth aspect, the present invention relates to a method for identifying a compound according to the first aspect of the invention, by screening a plurality of compounds and selecting a compound with the desired activity. The compound may have the capacity to modulate the interaction between hr44 and an in vivo binding partner.

Screening for in vivo Binding Partners for Hr44

Cellular proteins which interact with hr44 in vivo may be identified using a yeast two-hybrid assay. The assay is based on the finding that most eukaryotic transcription activators are modular, i.e., that the activators typically contain activation domains that activate transcription, and DNA binding domains that localise the activator to the appropriate region of a DNA molecule. In a two hybrid system, a first fusion protein contains one of a pair of interacting proteins (e.g. hr44) fused to a DNA binding domain, and a second fusion protein contains the other of a pair of interacting proteins (e.g. an in vivo binding partner) fused to a transcription activation domain. The two fusion proteins are independently expressed in the same cell, and interaction between the “interacting protein” portions of the fusions reconstitute the function of the transcription activation factor, which is detected by activation of transcription of a reporter gene. A plurality of candidate in vivo binding partners can be screened using the system, for example by expressing a cDNA library from an hr44-expressing tissue in the cell.

At least two different cell-based two hybrid protein-protein interaction assay systems have been developed. The yeast GAL4 two hybrid system was developed to detect protein-protein interaction based on the reconstitution of function of GAL4, a transcriptional activator from yeast, by activation of a GAL1-lacZ reporter gene. Like several other transcription activating factors, the GAL4 protein contains two distinct domains, a DNA binding domain and a transcription activation domain. Each domain can be independently expressed as a portion of a fusion protein composed of the domain, and a second, “bait” interacting protein. The two fusion proteins are then independently expressed together in a cell. When the two GAL4 domains are brought together by a binding interaction between the two “interacting” proteins, transcription of a reporter gene under the transcriptional control of GAL4 is initiated. The reporter gene typically has a promoter containing GAL4 protein binding sites (GAL upstream activating sequences, UASG).

A second two hybrid system utilises a native E. coli LexA repressor protein which binds tightly to appropriate operators. A plasmid is used to express one of a pair of interacting proteins (the “bait” protein) as a fusion to LexA. The plasmid expressing the LexA-fused bait protein is used to transform a reporter strain of yeast, such as EGY48. In this strain, binding sites for LexA are located upstream of two reporter genes. In the first reporter system, the upstream activation sequences of the chromosomal LEU2 gene—required in the biosynthetic pathway for leucine (Leu)—are replaced in EGY48 with lexA operators, permitting selection for viability when cells are plated on medium lacking Leu. In the second reporter system, EGY48 harbours a plasmid, pSH18-34, that contains a lexA operator-lacZ fusion gene, permitting discrimination based on colour when the yeast is grown on medium containing Xgal.

The present invention describes the use of the GAL4 two-hybrid system to detect four in vivo binding partners for hr44 (Example 1).

Screening for Compounds which Bind Hr44

A plurality of candidate compounds may be screened using the methods described below. In particular, these methods may be suited for screening libraries of compounds.

Where the candidate compounds are proteins, in particular antibodies or peptides, libraries of candidate compounds can be screened using phage display techniques. Phage display is a protocol of molecular screening which utilises recombinant bacteriophage. The technology involves transforming bacteriophage with a gene that encodes the library of candidate compounds, such that each phage or phagemid expresses a particular candidate compound. The transformed bacteriophage (which preferably is tethered to a solid support) expresses the appropriate candidate compound and displays it on their phage coat. Specific candidate compounds which are capable of interacting with hr44 are enriched by selection strategies based on affinity interaction. The successful candidate agents are then characterised. Phage display has advantages over standard affinity ligand screening technologies. The phage surface displays the candidate agent in a three dimensional configuration, more closely resembling its naturally occurring conformation. This allows for more specific and higher affinity binding for screening purposes.

The yeast two-hybrid system described above can be used to screen for polypeptides which bind hr44. For example, the human cDNA from a hr44-expressing tissue (which may be used to identify in vivo binding partners) may be substituted with a cDNA library from a different tissue or species, or a combinatorial library of synthetic oligonucleotides.

Another method of screening a library of compounds utilises eukaryotic or prokaryotic host cells which are stably transformed with recombinant DNA molecules expressing the library of compounds. Such cells, either in viable or fixed form, can be used for standard binding-partner assays. See also Parce et al. (1989) Science 246:243-247; and Owicki et al. (1990) Proc. Nat'l Acad. Sci. USA 87; 4007-4011, which describe sensitive methods to detect cellular responses. Competitive assays are particularly useful, where the cells expressing the library of compounds are incubated with a labelled antibody known to bind hr44, such as ¹²⁵I-antibody, and a test sample such as a candidate compound whose binding affinity to the binding composition is being measured. The bound and free labelled binding partners for hr44 are then separated to assess the degree of hr44 binding. The amount of test sample bound is inversely proportional to the amount of labelled anti-hr44 antibody binding to the hr44.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

Still another approach is to use solubilized, unpurified or solubilized purified hr44 either extracted from hr44-expressing mammalian cells or from transformed eukaryotic or prokaryotic host cells. This allows for a “molecular” binding assay with the advantages of increased specificity, the ability to automate, and high drug test throughput.

Another technique for candidate compound screening involves an approach which provides high throughput screening for new compounds having suitable binding affinity, e.g., to hr44, and is described in detail in International Patent application no. WO 84/03564 (Commonwealth Serum Labs.), published on Sep. 13, 1984. First, large numbers of different small peptide test compounds are synthesised on a solid substrate, e.g., plastic pins or some other appropriate surface; see Fodor et al. (1991). Then all the pins are reacted with solubilized hr44 and washed. The next step involves detecting bound hr44. Detection may be accomplished using a monoclonal antibody to hr44 (a number of which have already been prepared by the inventors using standard procedures). Compounds which interact specifically with hr44 may thus be identified.

Rational design of candidate compounds likely to be able to interact with hr44 may be based upon structural studies of the molecular shapes of hr44 and/or its in vivo binding partners. One means for determining which sites interact with specific other proteins is a physical structure determination, e.g., X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York. In particular, this would provide information on those regions of the hr44 polypeptide which are involved in homodimerisation, and interaction with pyruvate kinase, hnRNPE1, YP4 and fibrillarin and vice versa.

Screening for Compounds which Modulate the Activity of Hr44

As mentioned above, the compound may modulate the capacity of hr44 to interact with an in vivo binding partner of hr44. Once the in vivo binding partner has been identified, there are a number of methods known in the art by which compounds may be screened for their capacity to modulate the interaction between hr44 and its binding partner, or the physiological effect of the interaction.

For example, in vitro competitive binding assays using either immobilised hr44 or binding partner (see above) can be used to investigate the capacity of a library of test compounds to inhibit or enhance the hr44:binding partner interaction.

Alternatively, the yeast two-hybrid system as described above can be used to identify compounds which affect the hr44:binding partner interaction. For example, a first fusion protein (comprising the DNA binding domain of a transcription activating factor and hr44) and a second fusion protein (comprising the transcription activating domain and the binding partner) may be expressed in a yeast cell. When hr44:binding partner interaction takes place, transcription of a reporter gene under the transcriptional control of the transcriptional activator is initiated. Compounds which increase or decrease reporter expression relative to a user-defined threshold (for example, a five-fold increase or five-fold decrease) are thus identified as being modulators of the interaction.

The present inventors have found that the following proteins are in vivo binding partners for hr44: hnRNPE1; M1 and M2-type pyruvate kinase; YP4; and fibrillarin. The capacity of a compound to modulate the interaction between hr44 and any of these proteins may be measured by the standard techniques described above. Modulation of the interaction can also be measured by examining the changes in the physiological effect mediated by the interaction, as described below. Such physiological effects include: (i) metabolism of fatty acids, in particular linoleic acid or arachidonic acid; (ii) metabolism of pyruvate; and/or (iii) transport of prostaglandins.

The capacity of a compound to affect the interaction of hr44 with hnRNPE1 may be measured by determining 13-S-Hydroxyoctadecadienoic (13-HODE) or 15-hydroxyeicosatetraenoic acid (15-HETE) in a cellular assay. 13-S-HODE and 15-HETE are products of linoleic acid and arachidonic acid respectively, both produced by the enzymatic activity of 15-LOX1. The production of these metabolites may be quantified by ELISA (using commercially available reagents), by radioimmunoassays and/or gas chromatography.

The capacity of a compound to affect the interaction of hr44 with M2-type pyruvate kinase may be measured in cell culture assays by determining the concentrations of metabolites (e.g. pyruvate and lactate) by known methods (Huge F, et al 1992, J. of Cell. Physiol. 153:539-549; Bergmeyer, H U, editor, 1974, Methoden der enzymatischen Analyse, Vol I and II, 3rd ed., Verlag Chemie, Weinheim, Germany).

The capacity of a compound to interfere with binding may be measured by gel filtration as described earlier (Zwerschke W, et al, 1999, Proc. Natl. Acad. Sci. USA:96:1291-1296).

The interaction of hr44 with prostaglandin transporters and its effect on transport of prostaglandins, whether into or out of cells, may be measured using tritiated prostaglandins (available from NEN, LifeScience Products).

For comparative purposes, the screens for compounds which modulate the activity of hr44 may be performed in cultured tissue cells transfected with recombinant hr44, in non-transfected cells, and in cells treated with an hr44 anti-sense sequence.

Hr44 can interact with itself to form homodimers. Hr44 may be active (i.e. capable of interacting with an in vivo binding partner) or inactive in either of these two forms. Thus a compound may modulate the activity of hr44 by inhibiting or enhancing its capacity to dimerise. Compounds that interfere with dimerisation may be analysed by gel filtration methods (Zwerschke W. et al, 1999, Proc. Natl. Acad. Sci. USA 96:1291-1296).

Screening for Compounds which Modulate the Expression of Hr44

There are numerous methods suitable for measuring the expression of hr44, by measuring expression of the gene or the protein.

Hr44 gene expression may be measured using the polymerase chain reaction (PCR), for example using RT-PCR. RT-PCR may be a useful technique where the candidate compound is designed to block the transcription of the hr44 gene. Alternatively, the presence or amount of hr44 mRNA can be detected using Northern blot. Northern blotting techniques are particularly suitable if the candidate compound is designed to act by causing degradation of the hr44 mRNA. For example, if the candidate compound is an antisense sequence, which may cause the target mRNA to be degraded by enzymes such as RNAse H.

Hr44 protein expression may be detected or measures by a number of known techniques, including Western blotting, immunoprecipitation, immunocytochemisty techniques, immunohistochemistry, in situ hybridisation, ELISA, radio-immunolabelling, fluorescent labelling techniques (fluorimetry, confocal microscopy) and spectrophotometry.

Synthesis Methods

In a fifth aspect, the present invention provides a method for preparing a compound according to the first aspect of the invention.

For example, a compound capable of modulating the activity and/or expression of hr44 could be identified by the method of the fifth aspect of the invention, and then synthesised on a larger scale.

The methods appropriate to synthesise the identified compound will depend on its nature. For example, if the compound is a simple organic molecule, it may be synthesised using organic chemistry techniques. If the compound is a peptide, it may be synthesised using a peptide synthesiser. For longer peptides, polypeptides and proteins, it is usually easier to synthesise the compound using recombinant techniques, well known in the art. Alternatively, proteins may be isolated from source and polypeptides/peptides generated from them by protein degradation. Nucleic acids may be synthesised synthetically, or expressed from a gene. Where the successful candidate compound is a nucleic acid sequence, the compound can be synthesised by amplification from the candidate compound by known techniques (such as PCR).

Diagnostic Methods

According to an seventh aspect of the invention, there is provided a method of diagnosing a disease by detecting or measuring the expression of hr44.

Suitable methods by which the expression of the hr44 gene or protein may be measured are given above.

The diagnostic method of the eighth aspect of the invention is useful for diagnosing any disease which is associated with abnormal hr44 expression. For example, a particular disease may be associated with increased or decreased hr44 expression. Alternatively, the disease may be associated with an alteration in the distribution of hr44 expression, such as an altered intracellular, or tissue distribution.

In one embodiment, the disease is associated with an increase in hr44 expression in a localised area. The localised area may, for example be a tumour or an atherosclerotic lesion.

It is convenient if the expression of hr44 is measured by a non-invasive technique. The expression may be detected/measured in a body fluid or tissue after removal or excretion from the body. For example, the expression of hr44 may be detected in a sample of blood, saliva, urine or faeces from a patient. In this respect, hr44 is exported by cells and is known to be detectable in blood plasma, bile and pancreatic juice, so detection in blood samples and/or faeces is viable.

The monoclonal antibodies provided by the present invention (see Example 3) are particularly suitable for use in ELISA-type capture assays for the detection (quantification) of hr44 in body fluids.

The expression of hr44 is associated with a number of diseases, and can vary depending on the stage of the disease. Hence, expression of hr44 can be used as a diagnostic tool to indicate the stage to which a particular disease has progressed.

According to a eighth aspect of the invention, there is provided a method of monitoring the progression of a disease, which comprises the step of measuring the expression of hr44.

There are a number of methods known in the art by which the expression of hr44 may be measured, as explained above. Techniques such as immunohistology and in situ hybridisation are particularly suited to the method of the eighth aspect of the invention.

Examples of diseases which may be monitored by the method include cancer and vascular disease. In colon cancer, it has been found that the expression of hr44 is proportional to the size of the adenoma and thus is proportional to the rate of cell proliferation and/or diminished apoptosis in the formation of premalignant lesions. The expression of hr44 becomes variable with progression from well differentiated to poorly differentiated adenocarcinoma,

Polynucleotides and Polypeptides

According to a ninth aspect of the invention, there is provided a polynucleotide comprising the sequence shown as SEQ ID No. 1 or a derivative fragment, variant or homologue thereof.

The term “derivative, fragment, variant or homologue” in relation to the nucleotide SEQ ID No. 1 of the present invention includes any substitution of, modification of, replacement of, deletion of or addition of one (or more) nucleic acid from or to the sequence providing the resultant nucleotide sequence or the expression product thereof has the capability of binding to hr44. In particular, the term “homologue” covers homology with respect to function. With respect to sequence homology (i.e. similarity), preferably there is at least 75%, more preferably at least 85%, more preferably at least 90% homology to the sequence shown as SEQ ID No.1. More preferably there is at least 95%, such as at least 98%, homology to the sequence shown as SEQ ID No. 1.

According to a tenth aspect of the invention, there is provided a polypeptide comprising the sequence shown as SEQ ID No. 2 or a derivative fragment, variant or homologue thereof.

The term “derivative, fragment, variant or homologue” in relation to the protein SEQ ID No. 2 of the present invention includes any substitution of, modification of, replacement of, deletion of or addition of one (or more) amino acid from or to the sequence providing the resultant amino acid sequence has the capability of binding hr44. In particular, the term “homologue” covers homology with respect to function. With respect to sequence homology (i.e. similarity), preferably there is at least 75%, more preferably at least 85%, more preferably at least 90% homology to the sequence shown as SEQ ID No.2. More preferably there is at least 95%, such as at least 98%, homology to the sequence shown as SEQ ID No. 2.

### EXAMPLES

### Example 1

### Demonstration that Hr44 Interacts with Various Cytoplasmic and Nuclear Proteins

The proteins which interact with hr44 in vivo are analysed using an assay for detecting specific protein-protein interactions in yeast (such as the Matchmaker GAL4 Two-Hybrid system commercially available from Clontech). Hr44 (isolated from a human retina cDNA library, as described in Braun G. et al (1995) J. Exp. Med. 182:1121-1132) is subcloned into pGBDT7 and expressed in yeast Saccharomyces cerevisiae strain AH109. The transformed yeast strain is mated with a commercially available yeast strain (Saccharomyces cerevisiae strain Y187) that has been pre-transformed with a matchmaker cDNA library from human testis (Clontech laboratories). The cDNA from four of the isolated clones is isolated and sequenced. The FASTA and BLAST programs are used to compare identified cDNAs with entries of various DNA databases. The four clones are identified as expressing hnRNPE1, M2-type pyruvate kinase, fibrillarin and a novel protein YP4 which shares some identity with a prostaglandin transporter and organic anion transporter.

The Yeast Two Hybrid system also shows that hr44 forms homodimers. Using the Proscan sequence analysis package, hr44 is shown to contain a Myc-type helix-loop-helix dimerisation domain.

The partial DNA and amino acid sequence of YP4 was obtained using standard techniques and analysed using software available on www.expasy.ch/tools/or provided by the Human Genome Mapping Project at Hixton, Cambridge. The sequence and deduced primary structure of YP4 is shown as SEQ ID No. 1.

The interaction of hr44 with the above-mentioned proteins is confirmed by double transformations of yeast AH109 with pGBKT7-hr44 and the respective clones expressed from pACT2. Double transformations as controls are performed as follows: non-recombinant pGBKT7 together with the respective cDNA clones in pACT2 and pGBDT7-hr44 with non-recombinant pACT2.

### Example 2

### Investigating the Expression and Distribution of Hr44 Protein in Normal and Neoplastic Tissue

Specimens of normal and neoplastic human colon are taken and fixed with glutaraldehyde or formaldehyde. The expression of hr44 is determined by immunohistology using a monoclonal antibody produced against recombinant hr44. Staining in normal human colon shows that, in the epithelium, expression of hr44 is restricted to cells at the base of the crypt and to epithelial cells at the mucosal surface of the colon. By examining small, medium and large adenomas, it is shown that expression of hr44 is associated with increasing size of the adenoma. A high level of hr44 expression was detected in the epithelial cells forming the tumour in adenocarcinoma, although overall expression becomes more variable.

### Example 3

### The Identification and Characterisation of Compounds which Bind Hr44

Monoclonal antibodies to hr44 are produced and identified by the method described in Braun et al, 1995, J. Exp. Med. 182:1121-1132. In brief, following immunisation with recombinant hr44, hybridomas are produced using the P3×63 Ag8.653 cell line as a fusion partner for spleen cells from immunised Balb/c mice. The hybridomas are identified by ELISA using hr44. The monoclonal antibodies are designated 44/13A2, 44/31B2, 44/33A5, 44/33D3 and 44/72C2.

The hr44 epitopes recognised by the monoclonal antibodies are mapped as follows. A set of 83 peptides representing hr44 primary structure from the amino terminus to the carboxy terminus (purchased from Chiron Mimotopes Peptide Systems (Victoria, Australia)) is used for epitope mapping. The peptides consist of 12 amino acids, overlap by 8 residues, and are arranged on “pin-heads”. Analysis of mouse immune sera and monoclonal antibodies to hr44 is carried out in an ELISA type assay. To characterise conformational B-cell epitopes of hr44, a series of truncated fusions has been produced expressing various sized fragments of hr44. The monoclonals are tested by Western blot for their ability to recognise the truncated fusions.

The mAb 44/72C recognises a conformational B-cell epitope located between residues 100 and 219 of hr44.

The mAbs 44/13A2, 44/33A5,44/31B2 and 44/33D3 recognise linear peptide sequences of hr44. 44/13A2 recoginses a determinant contained within the sequence (YSTTPRIDEWRDKGYR; SEQ ID NO:3) at the amino-terminal of hr44. 44/33A5 recognises a linear epitope also near the amino-terminus within the sequence (EWRDKGYRLVED; SEQ ID NO:4). 44/31B2 recognises the epitope (NYDDNDDVEQIFIVKL; SEQ ID NO:5). 44/33D3 recognises a linear sequence (DVTPETPKTVDVTSETPKATPVKT; SEQ ID NO:6) at the carboxy terminus of hr44. This peptide contains a repeat sequence DVT(X)ETPK (SEQ ID NO:7) which has identified as the epitope recognised by 44/33D3.

### Example 4

### Screening for Compounds which Modulate the Interaction Between Hr44 and hnRNPE1 using a Competitive Binding Assay

In order to assess the capacity for the antibodies described in Example 3 to disrupt the interaction between hr44 and hnRNPE1, a competitive binding assay is conducted. Purified hr44 is coated on to the wells of a multi-well plate and left to adhere to the plastic. Excess hr44 solution is washed off, and the plate is treated with a solution containing bovine serum albumin (BSA) to block non-specific binding sites. ¹²⁵I-labelled hnRNPE1 solution is the added to each well and incubated for sufficient time to enable hr44:hnRNPE1 binding to occur. The cells are then washed thoroughly to remove and unbound ¹²⁵I-labelled hnRNPE1. Each well is then treated with a monoclonal antibody, or an equal volume of a solution containing an irrelevant protein control (BSA). After washing, the displacement of ¹²⁵I-labelled hnRNPE1 is measured using a plate reader. Antibodies which bound hr44 in such a way as to antagonise the hr44:hnRNPE1 interaction produced significantly fewer counts than control wells.

### Example 5

### Screening for Compounds which Modulate the Interaction Between Hr44 and hnRNPE1 using a Yeast Two-Hybrid Assay

The clone identified in Example 1 which is identified as expressing hnRNPE1 is used to identify compounds which are capable of disrupting the hr44:hnRNPE1 interaction. Cells from the clone are grown up and aliquots of the cells are treated in individual cells of a 96-well plate with individual compounds from a commercially available combinatorial library of small inorganic molecules, or are left untreated (control). Cells are transferred from a 96-well format on to Whatman filters (grade VWR). Freeze-thawing of the filters in liquid nitrogen is used to rupture the cells. The filters are then transferred on to another filter, pre-soaked in a di-sodium hydrogen phosphate/sodium di-hydrogen phosphate based buffer containing mercaptoethanol and 0.033% X-gal. β-Galactosidase producing cells can be identified by colour change to blue. Paler blue or white colonies indicate reduced reporter gene expression. The compounds with which these cells were treated are thus demonstrated as being capable of disrupting hr44:hnRNPE1 interaction.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

SEQ ID NO. 1

A partial sequence of the cDNA encoding YP4

| | |
|---|---|
| 1 | GAAGAGATGA TATTGAATAT TTTTCTCCCT GCTTTGCAGG GTGTACATAT |
| 51 | TCTAAAGCAC AAAACCAAAA AAAGATGTAC TACAATTGTT CTTGCATTAA |
| 101 | AGAAGGATTA ATAACTGCAG ATGCAGAAGG TGATTTTATT GATGCCAGAC |
| 151 | CCGGGAAATG TGATGCAAAG TGCTATAAGT TACCTTTGTT CATTGCTTTT |
| 201 | ATCTTTTCTA CACTTATATT TTCTGGTTTT TCTGGTGTAC CAATCGTCTT |
| 251 | GGCCATGACG CGGGTTGTAC CTGACAAACT GCGTTCTCTG GCCTTGGGTG |
| 301 | TAAGCTATGT GATTTTGAGA ATATTTGGGA CTATTCCTGG ACCATCAATC |
| 351 | TTTAAAATGT CAGGAGAAAC TTCTTGTATT TTACGGGATG TTAATAAATG |
| 401 | TGGACACAGA GGACGTTNTT GGATATATAA CAAGACAAAA ATGGCTTTCT |
| 451 | TAATGGTANG AATATGTTTC TTTGCAAACT AAGCACTATC ATCTTCACTA |
| 501 | CTATTGCATT TTCATATACA AACGTCGTCT AAATGAGAAC ACTGACTTCC |
| 551 | CAGATGTTAC TGTGAAGAAT CCCAAAAGTT AAAGAAAAAA GAAGAAACTG |
| 601 | GACTTGTAAC TGGATNAACA ATGNANTCTC NAAGATNTGG TTCTGTGNCC |
| 651 | AAACTTTCAA NAAGAGGAAA ATCACACATT AAGTTTACAT AAA1~TNGCAA |
| 701 | A1~ATNTATTT ATGGGGATCG GCATTTCAAN AATNAAAGTG TT |

SEQ ID No. 2

The cDNA (pos 1 to pos 417, see SEQ ID No. 1) encodes for following partial peptide sequence of YP4

| | |
|---|---|
| 1 | RDDIEYFSPC FAGCTYSKAQ NQKKMYYNCS CIKEGLITAD AEGDFIDARP |
| 51 | GKCDAKCYKL PLFIAFIFST LIFSGFSGVP IVLANTRVVP DKLRSLALGV |
| 101 | SYVILRIFGT IPGPSIFKMS GETSCILRDV NKCGHRGR |

1. A compound capable of modulating the activity, expression and/or subcellular organisation of hr44, for use in therapy.

2. A compound according to claim 1, which regulates alternative splicing of hr44 mRNA.

3. A compound according to claim 2, which is an antisense hr44 nucleic acid.

4. A compound according to claim 1, which is capable of modulating the interaction between hr44 and an in vivo binding partner.

5. A compound according to claim 4, which is capable of modulating the interaction between hr44 and any one or more of the following: hnRNPE1, YP4, M1-pyruvate kinase, M2-pyruvate kinase and fibrillarin.

6. A compound according to claim 1, which is capable of modulating hr44-associated alterations in metabolism.

7. A compound according to claim 4, which is capable of interacting specifically with a particular tissue-specific variant of hr44.

8. A compound according to claim 4, wherein the compound is an antibody.

9. A pharmaceutical composition comprising a compound according to claim 1 and a pharmaceutically acceptable carrier.

10. A compound comprising an anti-hr44 specific antibody and a conjugate molecule.

11. A compound according to claim 10, which directs the delivery of the composite molecule to one or more intracellular compartments.

12. A compound according to claim 10, wherein the compound is expressed by a DNA sequence comprising a sequence encoding an anti-hr44 antibody and a sequence encoding a conjugate compound.

13. A method of treating or preventing a disease in a subject, comprising administering a compound according to any of claims 1 to 8 or 10 to 12 to the subject.

14. A method of treating or preventing a disease in a subject, comprising administering a pharmaceutical composition according to claim 9 to the subject.

15. A method for treating or preventing a disease according to claim 13, comprising administering a compound which is capable of interacting specifically with a particular tissue-specific variant of hr44 to the subject, to target a particular tissue.

16. A method for treating or preventing a disease according to claim 13, wherein the disease is associated with an irregularity in fatty acid metabolism or transport; prostaglandin/anion metabolism or transport; glycolysis; lipogenesis; or a combination thereof

17. A method for treating or preventing a disease according to claim 13, wherein the disease is a vascular disease.

18. A method for treating or preventing a disease according to claim 13, wherein the disease is a cancer.

19. A method for identifying a compound useful in therapy, comprising
(i) screening a plurality of compounds; and
(ii) selecting a compound which binds to hr44 and/or modulates the activity and/or expression of hr44.

20. A method for preparing a compound useful in therapy, comprising
(i) screening a plurality of compounds;
(ii) selecting a compound which binds to hr44 and/or modulates the activity and/or expression of hr44; and
(iii) synthesising the selected compound.

21. A method of diagnosing a disease in a mammalian subject comprising detecting, or measuring the expression of hr44 in the subject.

22. A method according to claim 21, wherein hr44 is detected, or the expression of hr44 is measured, in a body fluid or tissue from the subject after removal or excretion from the body.

23. A method of monitoring the progression of a disease comprising measuring the expression of hr44.

24. A polynucleotide comprising the sequence shown as SEQ ID NO: 1 or a derivative, fragment, variant or homologue thereof.

25. A polypeptide comprising the sequence shown as SEQ ID NO: 2 or a derivative, fragment, variant or homologue thereof.

26. A diagnostic reagent for diagnosing the presence and/or the progression of a disease, which reagent assays hr44 levels in a biological sample.

27. A diagnostic reagent according to claim 26, which is an anti-hr44 antibody.

28. A diagnostic reagent according to claim 26 or claim 27, for the diagnosis of malignancies.

29. A tumour-specific anti-hr44 antibody.


SEQUENCE LISTING (7 sequences)

SEQ ID NO: 1 - DNA, 742 bp, Homo sapiens
  misc_feature (1)...(742): n = A,T,C or G
gaagagatgatattgaatatttttctccctgctttgcagggtgtacatattctaaagcacaaaaccaaaaaaagatgtactacaattgttcttgcattaaagaaggattaataactgcagatgcagaaggtgattttattgatgccagacccgggaaatgtgatgcaaagtgctataagttacctttgttcattgcttttatcttttctacacttatattttctggtttttctggtgtaccaatcgtcttggccatgacgcgggttgtacctgacaaactgcgttctctggccttgggtgtaagctatgtgattttgagaatatttgggactattcctggaccatcaatctttaaaatgtcaggagaaacttcttgtattttacgggatgttaataaatgtggacacagaggacgttnttggatatataacaagacaaaaatggctttcttaatggtangaatatgtttctttgcaaactaagcactatcatcttcactactattgcattttcatatacaaacgtcgtctaaatgagaacactgacttcccagatgttactgtgaagaatcccaaaagttaaagaaaaaagaagaaactggacttgtaactggatnaacaatgnantctcnaagatntggttctgtgnccaaactttcaanaagaggaaaatcacacattaagtttacataaantngcaaaaatntatttatggggatcggcatttcaanaatnaaagtgtt

SEQ ID NO: 2 - protein, 138 aa, Homo sapiens
RDDIEYFSPCFAGCTYSKAQNQKKMYYNCSCIKEGLITADAEGDFIDARPGKCDAKCYKLPLFIAFIFSTLIFSGFSGVPIVLAMTRVVPDKLRSLALGVSYVILRIFGTIPGPSIFKMSGETSCILRDVNKCGHRGR

SEQ ID NO: 3 - protein, 16 aa, Homo sapiens
YSTTPRIDEWRDKGYR

SEQ ID NO: 4 - protein, 12 aa, Homo sapiens
EWRDKGYRLVED

SEQ ID NO: 5 - protein, 16 aa, Homo sapiens
NYDDNDDVEQIFIVKL

SEQ ID NO: 6 - protein, 24 aa, Homo sapiens
DVTPETPKTVDVTSETPKATPVKT

SEQ ID NO: 7 - protein, 8 aa, Homo sapiens
  VARIANT (1)...(8): Xaa = Any Amino Acid
DVTXETPK